(19) United States
(12) Patent Application Publication
(10) Pub. No.: US 2016/0367470 A1
(43) Pub. Date: Dec. 22, 2016

(54) WATER-IN-OIL SUNSCREEN COMPOSITION HAVING ORGANIC SUNSCREEN ACTIVES

(71) Applicant: L'OREAL, Paris (FR)

(72) Inventors: Catherine CHIOU, Saddle Brook, NJ (US); Sara Robyn Muenz YOUSSEF, Middlesex, NJ (US)

(21) Appl. No.: 14/744,648

(22) Filed: Jun. 19, 2015

(51) Int. Cl. A61K 8/894; A61K 8/58; A61K 8/41; A61K 8/37; A61Q 17/04; A61K 8/31; A61K 8/35

(52) U.S. Cl. CPC A61K 8/894; A61K 8/31; A61K 8/585; A61K 8/35; A61K 8/37; A61Q 17/04; A61K 8/41; A61K 2800/40; A61K 2800/591; A61K 2800/805

(57) ABSTRACT

A sunscreen composition having an oil phase comprising (i) at least one silicone elastomer gel comprising an emulsifying crosslinked silicone elastomer having both silicone chain branches and alkyl chain branches swelled in a non-polar, volatile swelling agent having a Hansen solubility parameter, delta a, of less than or equal to 5 and (ii) greater than about 10% by weight of the sunscreen composition, of at least one organic sunscreen active. The sunscreen composition further includes an aqueous phase comprising water. The sunscreen composition is a homogenous, water-in-oil emulsion that is storage stable.

### FIELD OF THE INVENTION

The present invention is directed to sunscreen compositions and methods of using and producing same. More specifically, the present invention is directed to a sunscreen composition in the form of a water-in-oil inverse emulsion having an oil phase comprising a silicone elastomer gel comprising an emulsifying crosslinked silicone elastomer having both silicone chain branches and alkyl chain branches swelled in a non-polar, volatile swelling agent, at least one organic UV filter, optionally, a branched- and/or linear-type silicone emulsifier and an aqueous phase.

### BACKGROUND OF THE INVENTION

Conventional sunscreen products generally take the form of ultraviolet (UV)-filter compounds and/or particulate UV-screening compounds (collectively, “sunscreen actives”) that are solubilized, emulsified, or dispersed in a vehicle, which is topically applied to the skin.

Emulsifying silicone elastomers are traditionally employed in cosmetic products to impart unique features to the product, such as a smooth, silky feel. Emulsifying silicone elastomers are not compatible with organic sunscreen actives in conventional, non-polar elastomer-based emulsions due to the highly polar nature of most organic sunscreen actives. Water-in-oil (W/O) emulsions containing emulsifying silicone elastomers and organic sunscreen actives generally suffer from poor stability making them commercially unfeasible. This is evidenced by the lack of sunscreen products containing emulsifying silicone elastomers on the market, and the available few are in the form of a thin, milky lotion which requires shaking prior to application.

Thus, there remains a need to provide a storage stable sunscreen composition in the form of a water-in-oil (W/O) emulsion comprising emulsifying silicone elastomers which has a thick, creamy texture and provides a pleasant sensorial experience to consumers.

### BRIEF SUMMARY OF THE INVENTION

In an exemplary embodiment, a sunscreen composition in the form of a stable, pleasant feeling W/O emulsion is provided. The composition includes an aqueous phase and an oil phase. The oil phase includes at least one silicone elastomer gel comprising an emulsifying crosslinked silicone elastomer having both silicone chain branches and alkyl chain branches swelled in a non-polar, volatile swelling agent, an organic sunscreen active, and optionally a branched- and/or linear-type silicone emulsifier. The sunscreen composition provides a velvety soft, melting and caring sensation upon application to the skin.

In another exemplary embodiment, a sunscreen composition having an oil phase comprising (i) at least one silicone elastomer gel comprising an emulsifying crosslinked silicone elastomer having both silicone chain branches and alkyl chain branches swelled in a non-polar, volatile swelling agent having a Hansen solubility parameter of less than or equal to 5 and (ii) greater than about 10% by weight of the sunscreen composition, of at least one organic sunscreen active. The sunscreen composition further includes an aqueous phase comprising water. The sunscreen composition is a homogenous, water-in-oil emulsion that is storage stable.

In another exemplary embodiment, a method for preparing the sunscreen composition is provided involving mixing the above-disclosed ingredients to form the composition.

The present disclosure is also directed to a method for cosmetic treatment of keratinous tissues by applying the above-disclosed composition onto a surface of the keratinous tissue.

Other features and advantages of the present invention will be apparent from the following more detailed description of the preferred embodiment which illustrates, by way of example, the principles of the invention.

### DETAILED DESCRIPTION OF THE INVENTION

All numbers expressing quantities of ingredients and/or reaction conditions are to be understood as being modified in all instances by the term “about”, unless otherwise indicated.

“Keratinous tissue,” as used herein, includes, but is not limited to, skin, hair, and nails.

“W/O emulsion,” as used herein, includes a water phase dispersed in an oil phase, where the oil phase is a continuous phase.

“Homogenous” means having the visual appearance of being substantially uniform throughout, i.e., visually appears as a single phase emulsion.

“Grainy” means the visual appearance of a non-homogenous phase as a result of coalescing, settlement or other mechanism that results in an undesirable oily tactile feel. For example, while not so limited, a grainy composition may have a cottage cheese-like appearance.

“Sun Protection Factor” or SPF is a value expressed mathematically by the ratio of the irradiation time necessary to attain the erythemogenic threshold with the UV screening agent to the time necessary to attain the erythemogenic threshold without UV screening agent. SPF generally provides information about the skin's resistance to ultraviolet B (UVB) radiation from the sun. The SPF rating system has been developed to provide consumer guidance in selecting sunscreens.

“Critical wavelength” is an absorption spectrum of a sunscreen composition characterized by an index, namely a wavelength, where the integral of the spectral absorbance curve reached 90% of the integral from 290 nm to 400 nm. The critical wavelength is used to determine the breadth of UV protection.

In the present application the term “ambient temperature” means a temperature of about 25° C.

The sunscreen compositions and methods of the present invention can comprise, consist of, or consist essentially of the essential elements and limitations of the invention described herein, as well as any additional or optional ingredients, components, or limitations described herein or otherwise useful in personal care compositions intended for topical application to keratinous tissue.

Applicants have surprisingly discovered a storage stable W/O emulsion which provides a superior sensorial experience to consumers containing at least one organic sunscreen active and at least one silicone elastomer gel comprising an emulsifying crosslinked silicone elastomer having both silicone chain branches and alkyl chain branches swelled in a non-polar, volatile swelling agent. It has conventionally been known that organic sunscreen actives are incompatible in conventional, non-polar elastomer-based emulsions. Moreover, the resultant formulations impart desirable skin-feel, including, but not limited to, a velvety soft, melting and caring sensation upon application onto an end-user's skin.

An advantage of an embodiment of the present disclosure includes providing a W/O sunscreen composition for incorporating organic sunscreen actives in a non-polar elastomer-based emulsion. Another advantage of an embodiment of the present disclosure includes providing sunscreen compositions that provide improved skin-feel properties. Another advantage of an embodiment of the present disclosure is that organic sunscreens can be provided at high loadings, including greater than 10% by weight of the sunscreen composition while maintaining stability and having a desirable skin-feel.

The W/O emulsion of the sunscreen composition according to the present disclosure has a stable, white, glossy cream appearance. When the sunscreen composition is applied to the skin in a conventional way, the sunscreen composition has a velvety soft, melting and caring sensation.

Oil Phase

The oil phase present in the sunscreen composition, according to the disclosure, includes at least one silicone elastomer gel comprising an emulsifying crosslinked silicone elastomer having both silicone chain branches and alkyl chain branches swelled in a non-polar, volatile swelling agent, an organic sunscreen active, and optionally, a branched- and/or linear-type silicone emulsifier. The oil phase sunscreen composition is at a concentration, by weight, of about 10% to about 60%, or alternatively about 20% to about 50%, or alternatively about 30% to about 40%, based upon weight of the sunscreen composition.

Silicone Elastomer Gel

The emulsifying crosslinked silicone elastomer having both silicone chain branches and alkyl chain branches is swelled in a non-polar, volatile swelling agent. The resulted silicone elastomer-solvent mixture is in the form of a gel, herein as “silicone elastomer gel”. The amount of emulsifying crosslinked silicone elastomer present in the silicone elastomer gel is preferably in the amount of about 10% to about 80% by weight, more preferably in the amount of about 15% to about 60% by weight, and most preferably in the amount of about 20% to about 40% by weight, based on the total weight of the silicone elastomer gel.

The silicone elastomer gel is preferably present in the composition, according to the invention, in an amount of from about 0.5% to about 40%, such as from about 1% to about 20%, preferably from about 5% to about 10% by weight, based on the total weight of the composition.

Crosslinked Emulsifying Silicone Elastomer Having Silicone Chain and Alkyl Chain Branches

The silicone elastomer gel contains at least one crosslinked emulsifying silicone elastomer having both silicone chain branches and alkyl chain branches. Suitable examples include those known under the INCI name PEG-15/lauryl polydimethylsiloxyethyl dimethicone crosspolymer which is sold as a swollen composition comprising silicone oils or other oils, and commercially available under the tradenames KSG-320Z; KSG-350Z; KSG-380Z from Shin-Etsu Chemical Co. The methods of preparing such emulsifying silicone elastomers are disclosed by Sakuta and Tachibana in U.S. Pat. No. 8,592,547, which is hereby incorporated by reference.

A particularly suitable crosslinked emulsifying silicone elastomer having both silicone chain branches and alkyl chain branches for use with the present invention is PEG-15/lauryl polydimethylsiloxyethyl dimethicone crosspolymer.

Non-Polar Volatile Swelling Agent

The silicone elastomer gel includes the emulsifying crosslinked silicone elastomer having both silicone chain branches and alkyl chain branches swollen in a non-polar volatile swelling agent. For the purposes of the invention, the term “volatile swelling agent” means any oil that is capable of evaporating on contact with the skin in less than one hour, at room temperature and atmospheric pressure. The “volatile swelling agent” is a volatile cosmetic compound, which is liquid at room temperature, especially having a nonzero vapor pressure, at room temperature and atmospheric pressure, in particular having a vapor pressure ranging from 0.13 Pa to 40 000 Pa (10⁻³ to 300 mmHg), in particular ranging from 1.3 Pa to 13 000 Pa (0.01 to 100 mmHg) and more particularly ranging from 1.3 Pa to 1,300 Pa (0.01 to 10 mmHg). Suitable examples of non-polar volatile swelling agents may be hydrocarbon-based oils or silicone oils.

Among the volatile hydrocarbon-based oils containing from 8 to 16 carbon atoms, mention may be made especially of branched C₈-C₁₆ alkanes, for instance, C₈-C₁₆ isoalkanes (also known as isoparaffins), isododecane, isodecane, isohexadecane and, for example, the oils sold under the tradenames Isopar™ or Permethyl.

Mention may also be made of volatile linear alkanes comprising from 8 to 16 carbon atoms, in particular from 10 to 15 carbon atoms and more particularly from 11 to 13 carbon atoms, for instance, n-dodecane (C12) and n-tetradecane (C14) sold under the tradenames Parafol® 12-97 and Parafol 14-97® by the company Sasol, and the undecane-tridecane mixture, mixtures of n-undecane (C11) and of n-tridecane (C13) obtained sold under the tradename Cetiol® by the company BASF.

The solvents present in compositions, according to the present disclosure, are based upon solubility parameters disclosed by Charles Hansen in “Hansen Solubility Parameters: A User's Handbook” by Charles M. Hansen, CRC Press (2007). Each material is defined by three points in 3D space and these three points are known as the Hansen Solubility Parameters (HSP) which may be defined as follows.

Solubility parameters, as utilized herein, are calculated numerical constants which are a useful tool in predicting the behavior of a solvent material. There are three Hansen empirically- and theoretically-derived solubility parameters, a dispersion force component (δ_{d}), a polar or dipole interaction component (δₚ) and a hydrogen-bonding component (δₕ). Each of the three parameters (i.e., dispersion, polar and hydrogen bonding) represents a different characteristic of solvency, or solvent capability. In combination, the three parameters are a measure of the overall strength and selectivity of a solvent. The Total Hansen solubility parameter, which is the square root of the sum of the squares of the three parameters mentioned previously, provides a more general description of the solvency of the solvents. Individual and total Solubility Parameter units are given in (MPa)^{0.5}.

The global solubility parameter δ, according to the Hansen solubility space, is defined in the article “Solubility parameter values” by Eric A. Gruike in the book “Polymer Handbook”, 3rd Edition, Chapter VII, pp. 519-559, by the relationship:

δ²=δ_{d}²+δₚ²+δₕ²

in which:
δ_{d} characterizes the London dispersion forces derived from the formation of dipoles induced during molecular impacts,
δₚ characterizes the Debye interaction forces between permanent dipoles, and
δₕ characterizes the specific interaction forces (such as hydrogen bonding, acid/base, donor/acceptor, etc.).

The parameter δₐ, representing the polarity of a solvent molecule, is defined by the following relationship:

δₐ²=δₚ²+δₕ²=δ²−δ_{d}²

Volatile swelling agents for use in compositions, according to the present invention, include a polarity component of Hansen Solubility Parameter (δₐ) ranging from 0 to 5 (MPa)^{0.5}.

TABLE 1
Swelling Agent Solubility Factors

| | | | | | | | | Flash |
|---|---|---|---|---|---|---|---|---|
| | | | | | | η | | point |
| Swelling Agent | δ_{d} | δ_{ρ} | δₕ | δₐ | MW | (cPs) | Volatile | (° C.) |
| water | 15.5 | 16 | 42.3 | 45.22 | 18 | 1.00 | Yes | None |
| ethanol | 15.1 | 8.39 | 18.33 | 20.16 | 46 | 1.08 | Yes | 13 |
| isododecane | 15.05 | 0 | 0 | 0 | 170 | 1.4 | Yes | 43-45 |
| Undecane (and) tridecane | 15.54 | 0 | 0 | 0 | 170 | 1.3 | Yes | 81 |
| cyclotetradimethylsiloxane | 11.44 | 1.09 | 4.29 | 4.43 | 297 | 2.3 | Yes | 55 |
| (D4) | | | | | | | | |
| cyclopentadimethylsiloxane | 10.8 | 1.12 | 4.3 | 4.44 | 371 | 3.8 | Yes | 72-77 |
| (D5) | | | | | | | | |
| cyclohexadimethylsiloxane | 9.74 | 1.14 | 4.32 | 4.47 | 445 | 7.5 | Yes | 93 |
| (D6) | | | | | | | | |
| PDMS, 1 cst (L3) | 10.3 | 1.09 | 4.29 | 4.43 | 236 | 1.1 | Yes | 34 |
| PDMS, 1.5 cst (L4) | 10.3 | 1.09 | 4.29 | 4.43 | 310 | 1.5 | Yes | 64 |
| PDMS, 2 cst (L5) | 10.4 | 1.09 | 4.29 | 4.43 | 384 | 2 | Yes | 75 |
| PDMS, 6 cst (L10) | 10.68 | 2.09 | 4.14 | 4.64 | 770 | 4.96 | No | >101 |
| PDMS, 100 cst (L80) | 11.25 | 2.31 | 4.25 | 4.84 | 5,970 | 91 | No | >275 |
| PDMS, 300 cst (L180) | 11.31 | 2.31 | 4.26 | 4.85 | 13,650 | 288 | No | >300 |
| PDMS, 60,000 cst | 11.38 | 2.32 | 4.26 | 4.85 | 120,000 | 60,000 | No | >320 |

PDMS = Polydimethylsiloxane

Suitable swelling agents having linear silicone structures include, but are not limited to, hexamethyldisiloxane (L2), octamethyltrisiloxane (L3), decamethyltetrasiloxane (L4) and dodecamethylpentasiloxane (L5).

Suitable swelling agents having cyclic silicone structures include, but are not limited to, hexamethylcyclotrisiloxane (D3), octamethylcyclotetrasiloxane (D4), decamethylcyclopentasiloxane (D5), and dodecamethylcyclohexasiloxane (D6).

Particularly suitable non-polar volatile swelling agents for use with the present invention are dodecamethylpentasiloxane and isododecane.

A particularly suitable silicone elastomer gel is PEG-15/lauryl polydimethylsiloxyethyl dimethicone crosspolymer swelled in dodecamethylpentasiloxane or in isododecane.

Branched- and/or Linear-Type Silicone Co-Emulsifier

In one embodiment, the oil phase of the sunscreen composition optionally includes at least one branched- and/or linear-type silicone emulsifier. Suitable examples of branched- and/or linear-type silicone emulsifiers include, PEG-9 polydimethylsiloxyethyl dimethicone available under the tradename KF-6028 and lauryl PEG-9 polydimethylsiloxyethyl dimethicone available under the tradename KF-6038, both available from Shin-Etsu.

A preferred branched- and/or linear-type silicone emulsifier, according to the present invention, includes lauryl PEG-9 polydimethylsiloxyethyl dimethicone.

By way of illustration, a particularly suitable embodiment comprises a combination of emulsifying crosslinked siloxane elastomer and branched- and/or linear-type silicone emulsifier.

The branched- and/or linear-type silicone emulsifier is preferably present in the composition, according to the invention, in an amount of from about 0.1% to 5.0%, such as from about 0.5% to 4.0%, preferably from about 1% to 3.6% by weight, based on the total weight of the composition.

Organic Sunscreen Active

The sunscreen active agents are chosen especially from cinnamic derivatives; anthranilates; salicylic derivatives; dibenzoylmethane derivatives; camphor derivatives; benzophenone derivatives; diphenylacrylate derivatives; triazine derivatives; benzotriazole derivatives; benzalmalonate derivatives, especially those cited in U.S. Pat. No. 5,624,663; benzimidazole derivatives; imidazolines; bis-benzoazolyl derivatives as described in patents EP 669323 and U.S. Pat. No. 2,463,264; p-aminobenzoic acid (PABA) derivatives; methylene bis(hydroxyphenylbenzotriazole) derivatives as described in applications U.S. Pat. No. 5,237,071, U.S. Pat. No. 5,166,355, GB 2303549, DE 19726184 and EP 893119; benzoxazole derivatives as described in patent applications EP 0832642, EP 1027883, EP 1300137 and DE 10162844; screening polymers and screening silicones such as those described especially in patent application WO 93/04665; dimers derived from alkyl-styrene such as those described in patent application DE 19855649; 4,4-diarylbutadienes such as those described in patent applications EP 0967200, DE 19746654, DE 19755649, EP-A-1008586, EP 1133980 and EP 1133981, merocyanine derivatives such as those described in patent applications WO 04/006878, WO 05/058269 and WO 06/032741; and mixtures thereof, the entire contents of the patents and patent applications being incorporated by reference in their entirety.

As examples of other suitable organic sunscreen actives, mention may be made of those denoted hereinbelow under their INCI name:

Cinnamic Derivatives:

Examples of suitable cinnamic derviatives include, but are not limited to, ethylhexyl methoxycinnamate sold in particular under the tradename “Parsol® MCX” by DSM Nutritional Products, isopropyl methoxycinnamate, isoamyl methoxycinnamate sold under the tradename “Neo Heliopan® E 1000” by Symrise, DEA methoxycinnamate, diisopropyl methylcinnamate, glyceryl ethylhexanoate dimethoxycinnamate.

Dibenzoylmethane Derivatives:

Examples of suitable dibenzoylmethane derivatives include, but are not limited to, butyl methoxydibenzoylmethane sold especially under the tradename “Parsol® 1789” by DSM, isopropyl dibenzoylmethane.

Para-Aminobenzoic Acid Derivatives:

Examples of suitable para-aminobenzoic acid derivatives include, but are not limited to, PABA, ethyl PABA, ethyl dihydroxypropyl PABA, ethylhexyl dimethyl PABA sold in particular under the name Escalol™ 507 by ISP, Glyceryl PABA, PEG-25 PABA sold under the name “Uvinul® P25” by BASF.

Salicylic Derivatives:

Examples of suitable salicylic derivatives include, but are not limited to, homosalate sold under the name “Eusolex® HMS” by Rona/EM Industries, ethylhexyl salicylate sold under the name “Neo Heliopan® OS” by Symrise, dipropylene glycol salicylate sold under the name “Dipsal™” by Scher, TEA salicylate sold under the name “Neo Heliopan® TS” by Symrise.

Beta, Beta-Diphenylacrylate Derivatives:

Examples of suitable beta, beta-Diphenylacrylate derivatives include, but are not limited to, octocrylene sold in particular under the tradename “Uvinul® N539” by BASF, etocrylene sold in particular under the tradename “Uvinul® N35” by BASF.

Benzophenone Derivatives:

Examples of suitable benzophenone derivatives include, but are not limited to, benzophenone-1 sold under the tradename “Uvinul® 400” by BASF, benzophenone-2 sold under the tradename “Uvinul® D50” by BASF, benzophenone-3 or oxybenzone sold under the tradename “Uvinul® M40” by BASF, benzophenone-4 sold under the tradename “Uvinul® MS40” by BASF, benzophenone-5, benzophenone-6 sold under the tradename “Helisorb® 11” by Norquay, benzophenone-8 sold under the tradename “Spectra-Sorb UV-24” by American Cyanamid, benzophenone-9 sold under the tradename “Uvinul® DS-49” by BASF, benzophenone-12, n-hexyl 2-(4-diethylamino-2-hydroxybenzoyl)benzoate sold under the tradename “Uvinul® A+” or as a mixture with octyl methoxycinnamate under the tradename “Uvinul® A+B” by BASF.

Benzylidenecamphor Derivatives:

Examples of suitable benzylidenecamphor derivatives include, but are not limited to, 3-Benzylidene camphor manufactured under the name “Mexoryl™ SD” by Chimex, 4-methylbenzylidene vamphor sold under the name “Eusolex® 6300” by Merck, Benzylidene camphor sulfonic acid manufactured under the name “Mexoryl™ SL” by Chimex, camphor benzalkonium methosulfate manufactured under the name “Mexoryl™ SO” by Chimex, terephthalylidene dicamphor sulfonic acid manufactured under the name “Mexoryl™ SX” by Chimex, polyacrylamidomethyl benzylidene camphor manufactured under the name “Mexoryl™ SW” by Chimex.

Phenylbenzimidazole Derivatives:

Examples of suitable phenylbenzimidazole derivatives include, but are not limited to, phenylbenzimidazole sulfonic acid sold in particular under the tradename “Eusolex® 232” by Merck, disodium phenyl dibenzimidazole tetrasulfonate sold under the tradename “Neo Heliopan® AP” by Symrise.

Phenylbenzotriazole Derivatives:

Examples of suitable phenylbenzotriazole derivatives include, but are not limited to, drometrizole trisiloxane sold under the name “silatrizole” by Rhodia Chimie, methylene bis-benzotriazolyl tetramethylbutyl-phenol sold in solid form under the tradename “MIXXIM BB/100” by Fairmount Chemical, or in micronized form as an aqueous dispersion under the tradename “Tinosorb® M” by Ciba Specialty Chemicals.

Triazine Derivatives:

Examples of suitable triazine derivatives include, but are not limited to, bis-Ethylhexyloxyphenol methoxyphenyl triazine sold under the tradename “Tinosorb® S” by BASF, ethyihexyl triazone sold in particular under the tradename “Uvinul® T150” by BASF, diethyihexyl butamido triazone sold under the tradename “Uvasorb® HEB” by Sigma 3V, 2,4,6-tris(dineopentyl 4′-aminobenzalmalonate)-s-triazine, 2,4,6-tris(diisobutyl 4′-aminobenzalmalonate)-s triazine, 2,4-bis(dineopentyl 4′-aminobenzalmalonate)-6-(n-butyl 4′-aminobenzoate)-s-triazine, symmetrical triazine screening agents described in U.S. Pat. No. 6,225,467, patent application WO 2004/085412 (see compounds 6 and 9) or the document “Symmetrical Triazine Derivatives” IP.COM Journal, IP.COM Inc., West Henrietta, N.Y., US (20 Sep. 2004), especially 2,4,6-tris(biphenyl)-1,3,5-triazines (in particular 2,4,6-tris(biphenyl-4-yl)-1,3,5-triazine and 2,4,6-tris(terphenyl)-1,3,5-triazine, which is included in patent applications WO 06/035000, WO 06/034982, WO 06/034991, WO 06/035007, WO 2006/034992 and WO 2006/034985).

Anthranilic Derivatives:

An example of a suitable anthranilic derivative includes, but is not limited to, methyl anthranilate sold under the tradename “Neo Heliopan® MA” by Symrise.

Imidazoline Derivatives:

An example of a suitable imidazoline derivative includes, but is not limited to, ethylhexyl dimethoxybenzylidene dioxoimidazoline propionate.

Benzalmalonate Derivatives:

An example of a suitable Benzalmalonate derivative includes, but is not limited to, polyorganosiloxane containing benzalmalonate functions, for instance, polysilicone-15, sold under the tradename “Parsol® SLX” by DSM Nutritional Products.

4,4-Diarylbutadiene Derivatives:

An example of a suitable 4,4-diarylbutadiene derivative includes, but is not limited to, 1-Dicarboxy(2,2′-dimethylpropyl)-4,4-diphenyl-butadiene.

Benzoxazole Derivatives:

An example of suitable benzoxazole derivative includes, but is not limited to, 2,4-bis[5-(1-dimethyipropyl)benzoxazol-2-yl-(4-phenyl)imino]-6-(2-ethylhexyl)imino-1,3,5-triazine sold under the name Uvasorb® K2A by Sigma 3V, and mixtures thereof.

Particularly suitable organic sunscreen actives are selected from:

Ethylhexyl methoxycinnamate, ethylhexyl salicylate, homosalate, butyl methoxydibenzoylmethane, octocrylene, phenylbenzimidazole sulfonic acid, benzophenone-3, benzophenone-4, benzophenone-5, n-hexyl 2-(4-diethylamino-2-hydroxybenzoyl)benzoate, 4-methylbenzylidene camphor, terephthalylidene dicamphor sulfonic acid, disodium phenyl dibenzimidazole tetrasulfonate, methylene bis-benzotriazolyl tetramethylbutylphenol, bis-ethylhexyloxyphenol methoxyphenyl triazine, ethylhexyl triazone, diethylhexyl butamido triazone, 2,4,6-tris(dineopentyl 4′-aminobenzalmalonate)-s-triazine, 2,4,6-tris(diisobutyl 4′-aminobenzalmalonate)-s-triazine, 2,4-bis(dineopentyl 4′-aminobenzalmalonate)-6-(n-butyl 4′-aminobenzoate)-s-triazine, 2,4,6-tris(biphenyl-4-yl)-1,3,5-triazine, 2,4,6-tris(terphenyl)-1,3,5-triazine, drometrizole trisiloxane, polysilicone-15, 1,1-dicarboxy(2,2′-dimethylpropyl)-4,4-diphenylbutadiene, 2,4-bis[5-1(dimethylpropyl)benzoxazol-2-yl-(4-phenyl) [symbol] imino]-6-(2-ethylhexyl)imino-1,3,5-triazine, and mixtures thereof.

The organic sunscreen actives, according to the invention, are preferably present in amounts ranging from about 10% to about 40%, more preferably from about 10% to about 30% and even more preferably from about 14% to about 20% relative to the total weight of the sunscreen composition.

The present sunscreen composition, according to the present disclosure, includes a sun protection factor (SPF) of greater than or equal to 15 or SPF of at least about 15 to about 50 or SPF at least about 15 to about 30 and exhibits a critical wavelength of greater than 370 nm.

Aqueous Phase

The aqueous phase of the W/O emulsion sunscreen composition is at a concentration, by weight, of about 40% to about 90%, or alternatively about 50% to about 80%, or alternatively about 60% to about 70% based upon weight of the sunscreen composition. The aqueous phase present in the sunscreen composition, according to the disclosure, includes water and other water-soluble ingredients, such as, for example, pH-adjusting agents, glycols, lower alcohols (e.g. ethanol, propanediol), chelating agents, and preservatives.

Water-Soluble Active Ingredient

The aqueous phase present in the composition, according to the disclosure, includes at least one water-soluble active ingredient at a concentration, by weight, of from about 0.1% to about 20%, or alternatively from about 0.1% to about 15%, or alternatively from about 0.5% to about 10% based upon weight of the composition.

The water-soluble active ingredients can be present in their synthetic chemical compound forms, or alternatively as integral part of botanical extracts. Suitable examples of water-soluble ingredients, include, but are not limited to, (1) phenolic and polyphenolic compounds, and (2) other non-phenolic compounds.

The salts of the compounds that may be used, according to the invention, are chosen in particular from salts of alkali metals, for example, sodium or potassium; salts of alkaline-earth metals, for example, calcium, magnesium or strontium; metal salts, for example, zinc, aluminum, manganese or copper; salts of ammonium of formula NH4+; quaternary ammonium salts; salts of organic amines, for instance, salts of methylamine, dimethylamine, trimethylamine, triethylamine, ethylamine, 2-hydroxyethylamine, bis(2-hydroxyethyl)amine or tris(2-hydroxyethyl)amine; lysine or arginine salts. Salts chosen from sodium, potassium, magnesium, strontium, copper, manganese or zinc salts are preferably used. The sodium salt is preferentially used.

Phenolic and polyphenolic compounds include, but are not limited to, flavones, chalcones, tannins, phenolic acids, catechins, anthocyanidins, stilbenoids, curcuminoids, phenylpropanenoids. Many of phenolic and polyphenolic compounds are well-known antioxidants and/or compounds that can provide skin care and cosmetic benefits. Particularly suitable compounds include baicalin, resveratrol, ferulic acid, ellagic acid, salicylic acid, and botanical extracts.

Other non-phenolic, water-soluble compounds include, but are not limited to, vitamins, xanthines, ceramides, cholesterols, sphingosines, C-glycosides, zwitterionic N-substituted amino sulfonic acid buffers, jasmonic acid and derivatives, hyaluronic acid and derivatives, sugars, nucleic acids, α- and β-hydroxy acids, botanical extracts, aminopropyl triethoxysilane (APTES), dihydroxyacetone (DHA), amino acids, and peptides, and their derivatives and mixtures thereof.

Optional Hydrotropes

The composition of the present disclosure may optionally include hydrotropes. Examples of suitable hydrotropes include, for example, but are not limited to, nicotinamide, caffeine, sodium PCA, sodium salicylate, urea, or hydroxyethyl urea. At least one or a combination of two or more hydrotropes can be used to improve the solubility of phenolic and polyphenolic compounds in the water phase.

Hydrotropes may be present in the compositions in amounts generally ranging from about 0.1% to about 20% by weight, preferably from about 0.5% to about 10% by weight, and most preferably from about 1% to about 5% by weight, based on the total weight of the composition.

Optional Powders

The sunscreen composition of the present disclosure may optionally include cosmetic powders. The optional cosmetic powders provide formulas that are smoother and softer on the skin. Representative cosmetic powders include, but are not limited to, talc, mica, magnesium carbonate, calcium carbonate, magnesium silicate, aluminum magnesium silicate, silica, titanium dioxide, zinc oxide, red iron oxide, yellow iron oxide, black iron oxide, polyethylene powder, methacrylate powder, polystyrene powder, silk powder, crystalline cellulose, starch, titanated mica, iron oxide titanated mica, bismuth oxychloride, and the like. Additional powders include, but are not limited to, inorganic powders, such as gums, chalk, Fuller's earth, kaolin, sericite, muscovite, phlogopite, synthetic mica, lepidolite, biotite, lithia mica, vermiculite, aluminum silicate, starch, smectite clays, alkyl and/or trialkyl aryl ammonium smectites, chemically modified magnesium aluminum silicate, organically modified montmorillonite clay, hydrated aluminum silicate, fumed aluminum starch octenyl succinate barium silicate, calcium silicate, magnesium silicate, strontium silicate, metal tungstate, magnesium, silica alumina, zeolite, barium sulfate, calcined calcium sulfate (calcined gypsum), calcium phosphate, fluorine apatite, hydroxyapatite, ceramic powder, metallic soap (zinc stearate, magnesium stearate, zinc myristate, calcium palmitate, and aluminum stearate), colloidal silicone dioxide, and boron nitride; organic powder, such as polyamide resin powder (nylon powder), cyclodextrin, methyl polymethacrylate powder, copolymer powder of styrene and acrylic acid, benzoguanamine resin powder, poly(ethylene tetrafluoride) powder, and carboxyvinyl polymer, cellulose powder, such as hydroxyethyl cellulose and sodium carboxymethyl cellulose, ethylene glycol monostearate; inorganic white pigments, such as magnesium oxide. Representative cosmetic powders include, for example, polymethylsilsesquioxane, methyl polymethacrylate crosspolymer, Nylon-12, silica and boron nitride, and combinations thereof. Cosmetic powders may be present in the compositions in amounts generally ranging from about 0.1% to about 5% by weight or about 0.1% to about 10% by weight of the sunscreen composition.

The sunscreen composition of the present disclosure may also contain cosmetically acceptable additives or adjuvants as well as cosmetic or dermatologic active agents. Representative additives and adjuvants include, for example, water-soluble or water-miscible solvents or co-solvents or oil-soluble or oil-miscible solvents or co-solvents. Suitable examples of additives and adjuvants include, but are not limited to, fatty alcohols, fatty amides, alkylene carbonates, glycols, lower alcohols (e.g. ethanol, propanediol), dispersion enhancing agents, polymers, thickening agents, stabilizers, moisturizers, humectants, colorants, fillers, chelating agents, antioxidants (e.g. BHT, tocopherol), essential oils, fragrances, dyes, neutralizing or pH-adjusting agents (e.g., citric acid, triethylamine (TEA) and sodium hydroxide), preservatives, bactericides, conditioning or softening agents (e.g., panthenol and allantoin), extracts, such as botanical extracts, or any other ingredient commonly used in cosmetics for this type of application. Additives and adjuvants may be present in the compositions in amounts generally ranging from about 0.01% to about 10% by weight. Examples of cosmetic active agents or dermatological active agents include free-radical scavengers, vitamins (e.g., Vitamin E and derivatives thereof), anti-elastase and anti-collagenase agents, peptides, fatty acid derivatives, steroids, trace elements, extracts of algae and of planktons, enzymes and coenzymes, flavonoids and ceramides, hydroxy acids and mixtures thereof, and enhancing agents. These ingredients may be soluble or dispersible in any water phase(s) or oil phase(s) that is/are present in the sunscreen composition (i.e., aqueous and/or fatty (oil) phase).

Of course, a person skilled in the art will take care to choose this or these optional additional compounds so that the advantageous properties intrinsically attached to the sunscreen composition in accordance with the invention are not, or not substantially, detrimentally affected by the envisaged addition or additions.

Storage Stable

Compositions, according to the present disclosure, include a storage stable composition. By “storage stable” it is meant that the compositions initially have a good aesthetic appearance, including lack of a grainy texture and no visually perceptible separation of phases. In addition, the “storage stable” sunscreen composition, according to the present disclosure, is during extended storage including storage up to 12 weeks including exposure to a plurality of freeze/thaw cycles. For example, a sunscreen composition is considered storage stable if its viscosity is within 10-UD from the initial viscosity after 12 weeks storage at 25° C. and 45° C., after 10 freeze/thaw cycles (−20° C. to 25° C.) and does not exhibit signs of phase separation, does not show a grainy texture and/or become inhomogeneous.

Viscosity

Viscosity was measured at 25° C. using a Rheomat 180 equipped with a spindle rotating after 10 minutes at 200 rpm. Those skilled in the art may select the spindle for measuring the viscosity from spindles M3 or M4 on the basis of their general knowledge, so as to be able to perform the measurement. Viscosity is measured in UD (unit of deviation). Alternatively, viscosity measurements were taken on a Brookfield RVT Viscometer, using Heliopath spindles. The spindle was allowed to oscillate in the sample, and the measurements were taken after one minute.

Method

The sunscreen composition is prepared by combining the oil phase ingredients (shown as phase (A) in the examples), including organic UV filters, in a vessel and heating this oil phase while gently mixing until all solids dissolved, giving a homogeneous phase. The oil phase component is observed to verify that the components are dispersed and the mixing is continued while heating until fully homogeneous.

Once the oil phase components are mixed and homogenous, the water or aqueous phase ingredients (shown as phase (B) in the examples) are combined in a separate vessel and heated to, for example, 50-60° C. The water phase ingredients are mixed until the solids dissolved upon observation. In one embodiment, water-soluble UV filters can be added in the water phase.

Once the water phase is mixed and the components are dissolved, the water phase is added to the oil phase over a period of time, for example, for a period of 5-10 min. while mixing. The components are vigorously mixed, for example, with a propeller and/or rotor-stator, until a smooth and homogeneous emulsion is obtained.

In certain embodiments, an additional phase, such as a wax phase (shown as phase (C) in the examples), is mixed into the combined oil and water phases at a temperature above the melting temperatures of the waxes and subsequently cooled. Likewise, optionally, powders, fragrance and/or other temperature-sensitive ingredients can be added to the oil and water phases at lower temperatures and mixed well until smooth and homogeneous.

The following examples are intended to further illustrate the present invention. They are not intended to limit the invention in any way. Unless otherwise indicated, all parts are by weight.

### Examples

TABLE 2
Inventive Example

| | | Example 1 |
|---|---|---|
| Phase | US INCI NAME | % |
| A | ORGANIC SUNSCREEN ACTIVES | 15 |
| A | ISODODECANE (and) PEG-15/LAURYL | 10 |
| | POLYDIMETHYL-SILOXYETHYL | |
| | DIMETHICONE CROSSPOLYMER (75/25) | |
| | (KSG-320Z from Shin-Etsu, Akron, OH) | |
| A | LAURYL PEG-9 POLYDIMETHYLSILOXY- | 0.5 |
| | ETHYL DIMETHICONE | |
| | (KF-6038 from Shin-Etsu, Akron, OH) | |
| A | DISTEARDIMONIUM HECTORITE (and) | 2 |
| | PROPYLENE CARBONATE (10/3) | |
| A | ISOPROPYL LAUROYL SARCOSINATE | 6 |
| A | PRESERVATIVE | 0.15 |
| B | WATER | q.s. |
| B | GLYCERIN | 7 |
| B | WATER-SOLUBLE INGREDIENTS | 4.4 |
| C | POWDERS | 4 |
| | Total (%): | 100 |
| | Initial Viscosity (Mobile 3, 10 min) | 31.5 UD |
| | Initial Aspect | White Lotion |
| | 10 Cycles of Freeze/Thaw (−20° C. to 25° C.) | Pass |
| | Stability, 12 weeks | Pass |
| | 12 Weeks at 25° C. Viscosity (Mobile 3, 10 min) | 30.7 UD |
| | 12 Weeks at 45° C. Viscosity (Mobile 3, 10 min) | 25.8 UD |
| | Aspect, 12 weeks | White Lotion |

In making Example 1, the following procedure is used. The ingredients of phase A were combined in the main kettle and heated with gentle mixing until all the solids had dissolved and the mixture was homogeneous. The ingredients of phase B were combined in a beaker and heated to 50-60° C. with mixing until any solids had dissolved. Phase B was slowly added to phase A with mixing. Once the addition of phase B was complete, the batch was vigorously mixed until a smooth, homogenous emulsion was obtained. The temperature of the batch was allowed to cool and the ingredients of phase C were added with mixing until the batch was homogeneous.

The viscosity of the emulsion was measured after preparation. Samples of the emulsion were subjected to conditions of 25° C. and 45° C. for 12 weeks after which the viscosity was measured. Differences between the initial viscosity measurements and 12-week measurements showed the emulsion was stable. Samples of the emulsion were also subjected to 10 freeze/thaw cycles (−20° C. to 25° C. for a duration of 12 hours at each temperature).

TABLE 3
Inventive Examples

| | | Ex. 2 | Ex. 3 | Ex. 4 |
|---|---|---|---|---|
| Phase | US INCI Name | % | % | % |
| A | ORGANIC SUNSCREEN ACTIVES | 14 | 14 | 14 |
| A | DIMETHICONE (2 cSt) (AND) | 7 | 7 | 7 |
| | PEG-15/LAURYL | | | |
| | POLYDIMETHYLSILOXYETHYL | | | |
| | DIMETHICONE CROSSPOLYMER | | | |
| | (72/28) (KSG-380Z from | | | |
| | Shin-Etsu, Akron, OH) | | | |
| A | LAURYL PEG-9 | 1 | 1 | 1 |
| | POLYDIMETHYLSILOXYETHYL | | | |
| | DIMETHICONE | | | |
| | (KF-6038 from Shin-Etsu, | | | |
| | Akron, OH) | | | |
| A | SILICA SILYLATE | 0 | 0.8 | 0 |
| A | ACRYLATES/STEARYL | 1 | 1 | 1 |
| | ACRYLATE/DIMETHICONE | | | |
| | METHACRYLATE COPOLYMER | | | |
| A | ORBIGNYA OLEIFERA SEED OIL | 2 | 2 | 2 |
| A | DIISOPROPYL SEBACATE | 2 | 2 | 2 |
| A | DICAPRYLYL CARBONATE | 2 | 2 | 2 |
| A | PRESERVATIVE | 0.15 | 0.15 | 1.15 |
| B | WATER | q.s. | q.s. | q.s. |
| B | GLYCERIN | 7 | 7 | 7 |
| B | WATER-SOLUBLE INGREDIENTS | 4.8 | 4.8 | 4.8 |
| C | POLYETHYLENE | 2 | 2 | 2 |
| D | FRAGRANCE | 0.3 | 0.3 | 0.3 |
| D | DIMETHICONE (2 cst) | 3 | 3 | 3 |
| E | POWDERS | 2 | 2 | 4 |
| | Total (%): | 100 | 100 | 100 |
| | Initial Viscosity (Mobile 4, | 26.4 | 39.8 | 23.6 |
| | 10 min) (UD) | | | |
| | Initial Aspect | White Cream | | |
| | 10 Cycles of Freeze/Thaw | Pass | Pass | Pass |
| | (−20° C. to 25° C.) | | | |
| | Stability, 12 weeks | Pass | Pass | Pass |
| | 12 Weeks at 25° C. Viscosity | 20.2 | 34.2 | 20.7 |
| | (Mobile 4, 10 min) (UD) | | | |
| | 12 Weeks at 45° C. Viscosity | 19.4 | 30.7 | 32.7 |
| | (Mobile 4, 10 min) (UD) | | | |

| | |
|---|---|
| Aspect, 12 weeks | White Cream |

The Examples in Table 3 were prepared in a similar manner to Example 1. After phases A and B were combined, the ingredients of phase C were added, the temperature of the batch was increased, and mixing was continued until all the waxes were melted. Once the mixture was homogenous, the batch was vigorously mixed until a smooth, homogenous emulsion was obtained. The temperature of the batch was allowed to cool and the ingredients of phases D and E were added with mixing until the batch was homogeneous.

The viscosity of the emulsion was measured after preparation. Samples of the emulsion were subjected to conditions of 25° C. and 45° C. for 12 weeks after which the viscosity was measured. Differences between the initial viscosity measurements and 12-week measurements showed the emulsion was stable. Samples of the emulsion were also subjected to 10 freeze/thaw cycles (−20° C. to 25° C. for a duration of 12 hours at each temperature).

TABLE 4
Both Silicone and Alkyl Chain Branches
vs. Only Alkyl Chain Branches

| | | | Example 6 |
|---|---|---|---|
| | | Example 5 | (Compar- |
| | | (Inventive) | ative) |
| Phase | INCI US Name | % | % |
| A | ORGANIC SUNSCREEN | 13 | 13 |
| | ACTIVES | | |
| A | POLYGLYCERYL-4 | 1 | 1 |
| | ISOSTEARATE | | |
| A | DICAPRYLYL ETHER | 3 | 3 |
| A | DIMETHICONE (2 cSt) (and) | 5 | 0 |
| | PEG-15/LAURYL | | |
| | POLYDIMETHYLSILOXYETHYL | | |
| | DIMETHICONE CROSSPOLYMER | | |
| | (72/28) (KSG-380Z from | | |
| | Shin-Etsu, Akron, OH) | | |
| A | ISODODECANE (and) | 0 | 5 |
| | PEG-15/LAURYL | | |
| | DIMETHICONE CROSSPOLYMER | | |
| | (75/25) (KSG-320 from | | |
| | Shin-Etsu, Akron, OH) | | |
| A | DIMETHICONE (6 cSt) (and) | 3.6 | 3.6 |
| | DIMETHICONE/PEG-10/15 | | |
| | CROSSPOLYMER (75/25) | | |
| | (KSG-210 from Shin-Etsu, | | |
| | Akron, OH) | | |
| B | WATER | q.s. | q.s. |
| B | GLYCERIN | 15 | 15 |
| B | WATER-SOLUBLE INGREDIENTS | 4.3 | 4.3 |
| C | POWDERS | 2 | 2 |
| | Total (%): | 100 | 100 |
| | Initial Viscosity (Mobile 4, | 31.2 UD | 6.6 UD |
| | 10 min): | | |
| | Initial Aspect | White | White |
| | | Cream | Lotion |
| | 10 Cycles of Freeze/Thaw | Pass | Fail |
| | (−20° C. to 25° C.) | | (grainy |
| | | | texture) |

The Examples shown in Table 4 demonstrate the criticality of the use of a silicone elastomer having both silicone- and alkyl-chain branches. The compositions of Examples 5 and 6 are identical with the exception of the silicone elastomer used. In Example 5, a silicone elastomer, according to the invention, having both silicone- and alkyl-chain branches was used as the primary emulsifier. In Example 6, the silicone elastomer, according to the invention, was substituted with PEG-15/lauryl dimethicone crosspolymer (KSG-320 from Shin-Etsu), an emulsifying silicone elastomer having only alkyl-chain branches. After 10 freeze/thaw cycles (−20° C. to 25° C.), Example 5 maintained its smooth cream texture while Example 6 became grainy and was deemed unstable.

TABLE 5
Inventive Examples - Photoprotection

| | Ex. 4 | Ex. 5 | Ex. 11 |
|---|---|---|---|
| | (Inventive) | (Inventive) | (Inventive) |
| Critical Wavelength | 379.1 nm | 380 nm | 378 nm |
| In vivo SPF (FDA) | 18 | 24 | 18 |
| ISO 24444 (SPF) | 22.2 | NA | NA |
| ISO 24442 (UVA PF) | 10.7 | NA | NA |

TABLE 6
Comparative Examples

| | | Example 7 | Example 8 |
|---|---|---|---|
| Phase | INCI US Name | % | % |
| A | ORGANIC SUNSCREEN ACTIVES | 14 | 14 |
| A | DIMETHICONE (6 cSt) (and) | 7 | 0 |
| | DIMETHICONE/PEG-10/15 | | |
| | CROSSPOLYMER (75/25) | | |
| | (KSG-210 from Shin-Etsu, | | |
| | Akron, OH) | | |
| A | DIMETHICONE (6 cSt) (and) | 0 | 10 |
| | PEG-15/LAURYL | | |
| | POLYDIMETHYLSILOXYETHYL | | |
| | DIMETHICONE CROSSPOLYMER | | |
| | (72/28) (KSG-360Z from | | |
| | Shin-Etsu, Akron, OH) | | |
| A | LAURYL PEG-9 | 2 | 2 |
| | POLYDIMETHYLSILOXYETHYL | | |
| | DIMETHICONE | | |
| | (KF-6038 from Shin-Etsu, | | |
| | Akron, OH) | | |
| A | DISTEARDIMONIUM HECTORITE | 2 | 0 |
| | (and) PROPYLENE CARBONATE (10/3) | | |
| A | ACRYLATES/STEARYL | 1 | 1 |
| | ACRYLATE/DIMETHICONE | | |
| | METHACRYLATE COPOLYMER | | |
| A | ORBIGNYA OLEIFERA SEED OIL | 2 | 2 |
| A | DIISOPROPYL SEBACATE | 2 | 2 |
| A | PRESERVATIVE | 0.15 | 0.15 |
| B | WATER | q.s. | q.s. |
| B | GLYCERIN | 7 | 7 |
| B | WATER-SOLUBLE INGREDIENTS | 4.8 | 4.8 |
| C | HYDROGENATED JOJOBA OIL | 2 | 2 |
| C | SYNTHETIC WAX | 2 | 4 |
| D | FRAGRANCE | 0.3 | 0.3 |
| D | DIMETHICONE (2 cSt) | 5 | 5 |
| E | POWDERS | 4.7 | 5 |
| | Total (%): | 100 | 100 |
| | Initial Viscosity (Mobile 4, 10 min) | 21.8 UD | 34.9 UD |
| | Initial Aspect | White Cream | |
| | 10 Cycles of Freeze/Thaw | Pass | Pass |
| | (−20° C. to 25° C.) | | |
| | Stability, 12 weeks | Oil Phase Separation | |
| | 12 Weeks at 25° C. Viscosity | 20.0 UD | 31.2 UD |
| | (Mobile 4, 10 min) | | |
| | 12 Weeks at 45° C. Viscosity | 22.1 UD | 31.6 UD |
| | (Mobile 4, 10 min) | | |
| | Aspect, 12 weeks | Grainy | Very |
| | | | grainy |

Comparative Example 7 employed a conventional linear, emulsifying silicone elastomer (dimethicone/PEG-10/15 crosspolymer, KSG-210) having no alkyl-chain branches. Example 7 was initially a smooth, white cream, but, after 12 weeks, the oil phase separated and formed a layer on top, and the sample became grainy resulting in an oily/greasy texture. Therefore, Example 7 was deemed unstable and unacceptable.

Comparative Example 8 employed a silicone elastomer, according to the invention, having both silicone- and alkyl-chain branches. In comparison, the elastomer was swelled in a non-volatile dimethicone versus a volatile dimethicone, according to the invention, (6 cSt vs. 2 cSt). Example 8 was initially a smooth, white cream, but, after 12 weeks, the oil phase separated and formed a layer on top, and the sample became very grainy resulting in an oily/greasy texture. Therefore, Example 8 was deemed unstable and unacceptable.

TABLE 7
Comparative Examples

| | | Ex. 9 | Ex. 10 |
|---|---|---|---|
| | | (Compar- | (Compar- |
| | | ative) | ative) |
| Phase | INCI US Name | % | % |
| A | BUTYL METHOXYDIBENZOYL- | 2 | 2 |
| | METHANE | | |
| A | HOMOSALATE | 4 | 4 |
| A | ETHYLHEXYL SALICYLATE | 0 | 2 |
| A | OCTOCRYLENE | 1.25 | 1.25 |
| A | Isododecane (and) PEG-15/Lauryl | 2.5 | 2.5 |
| | Dimethicone Crosspolymer | | |
| | (75/25) (1) | | |
| A | CETYL PEG/PPG-10/1 | 1 | 0.5 |
| | DIMETHICONE | | |
| A | Triethylhexanoin (and) Vinyl | 0 | 4 |
| | Dimethicone/Lauryl Dimethicone | | |
| | Crosspolymer (70/30) (2) | | |
| A | ISOPROPYL LAUROYL | 2 | 2 |
| | SARCOSINATE | | |
| A | ISONONYL ISONONANOATE | 4 | 4 |
| A | ISOPROPYL ISOSTEARATE | 4 | 2 |
| A | DEXTRIN PALMITATE | 0.3 | 0 |
| A | ETHYLPARABEN | 0.1 | 0.1 |
| A | PROPYLPARABEN | 0.1 | 0.1 |
| A | POLYMETHYLSILSESQUIOXANE | 3 | 3 |
| B1 | WATER | 10 | 0 |
| B1 | TRIETHANOLAMINE | 0.55 | 0 |
| B1 | PHENYLBENZIMIDAZOLE | 1 | 0 |
| | SULFONIC ACID | | |
| B2 | WATER | 51 | 57.35 |
| B2 | Niacinamide | 4 | 4 |
| B2 | Sodium Citrate | 0.2 | 0.2 |
| B2 | Sodium Chloride | 0.5 | 0.5 |
| B2 | GLYCERIN | 2 | 7 |
| B2 | PENTYLENE GLYCOL | 3 | 3 |
| B2 | BUTYLENE GLYCOL | 3 | 0 |
| B2 | BENZYL ALCOHOL | 0.4 | 0.4 |
| B2 | DISODIUM EDTA | 0.1 | 0.1 |
| | Total (%): | 100 | 100 |
| | Initial Viscosity (Mobile 4, 10 min): | 18.3 UD | 13.1 UD |

| | |
|---|---|
| Initial Aspect: | White Lotion |
| 10 Cycles of Freeze/Thaw | Grainy appearance, both |
| (−20° C. to 25° C.): | formulas failed stability |

(1) KSG320 from Shin-Etsu, Akron, OH.
(2) KSG43 from Shin-Etsu, Akron, OH.

Comparative Examples 9 and 10 were prepared according to Examples 3 and 4 of US 2007/0274932 A1, assigned to The Procter & Gamble Company, respectively. Both Examples demonstrate the use of emulsifying silicone elastomer with an alkyl substitution and linear dimethicone crosspolymer (swelled in isododecane), while Example 10 uses an additional non-emulsifying silicone elastomer. Both Examples contain less than 10% UV filters, giving low UV photoprotection benefit. Upon being exposed to the freeze/thaw stability protocol, both formulas exhibited a “grainy” and inhomogeneous appearance, indicating an inherent instability of the formulas. Upon examination under microscope, the resulting emulsions from Examples 9 and 10 exhibited large areas of detachment along the border of the emulsion in addition to plural phase separation, all indicating instability of the emulsions.

TABLE 8
Inventive Example

| | | Ex. 11 |
|---|---|---|
| | | (Inventive) |
| Phase | INCI US Name | % |
| A | BUTYL METHOXYDIBENZOYLMETHANE | 3 |
| A | HOMOSALATE | 5 |
| A | ETHYLHEXYL SALICYLATE | 5 |
| A | OCTOCRYLENE | 3 |
| A | PEG-15/Lauryl Polydimethylsiloxyethyl | 5 |
| | Dimethicone Crosspolymer | |
| | (and) Dimethicone (28/72) (1) | |
| A | LAURYL PEG-9 | 1 |
| | POLYDIMETHYLSILOXYETHYL | |
| | DIMETHICONE (2) | |
| A | C30-45 ALKYLDIMETHYLSILYL | 2 |
| | POLYPROPYLSILSESQUIOXANE | |
| | (and) PARAFFIN | |
| A | DEXTRIN PALMITATE | 0.7 |
| A | DICAPRYLYL CARBONATE | 7 |
| A | TOCOPHEROL | 0.25 |
| A | SILICA SILYLATE | 0.5 |
| B1 | WATER | 41.15 |
| B1 | ADENOSINE | 0.1 |
| B1 | NIACINAMIDE | 3 |
| B1 | CAFFEINE | 2 |
| B1 | SODIUM METABISULFITE | 0.1 |
| B1 | RESVERATROL | 0.25 |
| B1 | CHLORPHENESIN | 0.3 |
| B1 | p-ANISIC ACID | 0.15 |
| B2 | GLYCERIN | 7 |
| B2 | PROPANEDIOL | 3 |
| B2 | PHENOXYETHANOL | 0.7 |
| B2 | DISODIUM EDTA | 0.1 |
| B2 | Sodium Citrate | 0.2 |
| B2 | Sodium Chloride | 0.5 |
| C | METHYL METHACRYLATE CROSSPOLYMER | 2 |
| C | NYLON-12 | 2 |
| D | ISODODECANE | 5 |
| | Total (%): | 100 |
| | Initial Viscosity (Mobile 3, 10 min): | 67.3 UD |
| | Initial Aspect: | White Lotion |
| | 10 Cycles of Freeze/Thaw | Pass |
| | (−20° C. to 25° C.): | |
| | Stability, 12 weeks | Pass |
| | 12 Weeks at 25° C. Viscosity | 60.7 UD |
| | (Mobile 3, 10 min): | |
| | 12 Weeks at 45° C. Viscosity | 61.3 UD |
| | (Mobile 3, 10 min): | |
| | Aspect, 12 weeks | White Lotion |

(1) KSG380Z from Shin-Etsu, Akron, OH.
(2) KF6038 from Shin-Etsu, Akron, OH.

The Example 11 shown in Table 8 demonstrates the inclusion of water-soluble actives into the composition while maintaining stability.

While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A sunscreen composition comprising:
(a) an oil phase comprising:
(i) at least one silicone elastomer gel comprising an emulsifying crosslinked silicone elastomer having both silicone chain branches and alkyl chain branches swelled in a non-polar, volatile swelling agent having a Hansen solubility parameter, delta a, of less than or equal to about 5;
(ii) greater than about 10% by weight of the sunscreen composition, of at least one organic sunscreen active; and,
(b) an aqueous phase comprising water; and
wherein the sunscreen composition is a homogenous, water-in-oil emulsion that is storage stable.

2. The composition of claim 1 wherein the emulsifying crosslinked silicone elastomer is PEG-15/lauryl polydimethylsiloxyethyl dimethicone crosspolymer.

3. The composition of claim 1 wherein the non-polar, volatile swelling agent is isododecane.

4. The composition of claim 1 wherein the non-polar, volatile swelling agent is dodecamethylpentasiloxane.

5. The composition of claim 1 wherein (i) is present in an amount of from about 0.5% to about 40% by weight, based on the total weight of the sunscreen composition.

6. The composition of claim 5 wherein (i) is present in an amount of from about 1% to about 20% by weight, based on the total weight of the sunscreen composition.

7. The composition of claim 6 wherein (i) is present in an amount of from about 5% to about 10% by weight, based on the total weight of the sunscreen composition.

8. The composition of claim 1 wherein the emulsifying crosslinked silicone elastomer is present in an amount of 10% to about 80% by weight, based on total weight of the silicone elastomer gel.

9. The composition of claim 1 wherein the emulsifying crosslinked silicone elastomer is present in an amount of 15% to about 60% by weight, based on total weight of the silicone elastomer gel.

10. The composition of claim 1 wherein the emulsifying crosslinked silicone elastomer is present in an amount of 20% to about 40% by weight, based on total weight of the silicone elastomer gel.

11. The composition of claim 1 further comprising a branched- and/or linear-type silicone emulsifier present in an amount of from about 0.1% to about 5.0% by weight, based upon the total weight of the sunscreen composition.

12. The composition of claim 1, wherein the at least one organic sunscreen active is selected from the group consisting of methyl anthranilate; cinnamic derivatives selected from the group consisting of ethylhexyl methoxycinnamate, isopropyl methoxycinnamate, isoamyl methoxycinnamate, cinoxate, DEA methoxycinnamate, diisopropyl methylcinnamate, glyceryl ethylhexanoate and dimethoxycinnamate; dibenzoylmethane derivatives selected from the group consisting of butyl methoxydibenzoylmethane and isopropyl dibenzoylmethane; salicylic derivatives selected from the group consisting of homosalate, ethylhexyl salicylate, dipropylene glycol salicylate, and TEA salicylate; camphor derivatives selected from the group consisting of 3-benzylidene camphor, 4-methylbenzylidene camphor, benzylidene camphor sulphonic acid, camphor benzalkonium methosulphate, terephthalylidene dicamphor sulphonic Acid, and polyacrylamidomethyl benzylidene camphor; triazine derivatives selected from the group consisting of anisotriazine, ethylhexyl triazone, 2,4,6-tris(diisobutyl 4′-aminobenzalmalonate)-s-triazine, and diethylhexyl butamido triazone; benzophenone derivatives selected from the group consisting of benzophenone-1, benzophenone-2, benzophenone-3, benzophenone-4, benzophenone-5, benzophenone-6, benzophenone-8, benzophenone-9, benzophenone-12, and n-hexyl 2-(4-diethylamino-2-hydroxybenzoyl)benzoate; β,β-diphenylacrylate derivatives selected from the group consisting of octocrylene and etocrylene; benzotriazole derivatives selected from the group consisting of drometrizole trisiloxane and methylene bis-benzotriazolyl tetramethylbutyiphenol; polysilicone-15; benzimidazole derivatives selected from the group consisting of phenylbenzimidazole sulphonic acid, and disodium phenyl dibenzimidazole tetra-sulphonate; ethyihexyl dimethoxybenzylidene dioxoimidazoline propionate; p-aminobenzoic acid (PABA) derivatives selected from the group consisting of PABA, ethyl PABA, ethyl dihydroxypropyl PABA, ethylhexyl dimethyl PABA, Glyceryl PABA and PEG-25 PABA; 2,4-bis[5-(dimethylpropyl)benzoxazol-2-yl(4-phenyl)imino]-6-(2-ethylhexyl)imino-1,3,5-triazine; polymer sunscreens and silicone sunscreens; dimers derived from α-alkylstyrene; 4,4-diarylbutadienes and combinations thereof.

13. The composition of claim 1, wherein the at least one organic sunscreen active is selected from the group consisting of homosalate, ethyihexyl salicylate, butyl methoxydibenzoylmethane, octocrylene, ethylhexyl methoxycinnamate, phenylbenzimidazole sulphonic acid, benzophenone-3, benzophenone-4, benzophenone-5, n-hexyl 2-(4-diethylamino-2-hydroxybenzoyl)benzoate, 4-methylbenzylidene camphor, terephthalylidene dicamphor sulphonic acid, disodium phenyl dibenzimidazole tetra-sulphonate,2,4,6-tris(diisobutyl 4′-aminobenzalmalonate)-s-triazine, anisotriazine, ethyihexyl triazone, diethylhexyl butamido triazone, tethylene bis-benzotriazolyl tetramethylbutylphenol, drometrizole trisiloxane, polysilicone-15,1,1-dicarboxy(2,2N-dimethylpropyl)-4,4-diphenylbutadiene,2,4-bis[5-(dimethylpropyl)benzoxazol-2-yl-(4-phenyl)imino]-6-(2-ethylhexyl)imino-1,3,5-triazine, and combinations thereof.

14. The composition of claim 1, wherein the at least one organic sunscreen active is selected from the group consisting of octocrylene, butyl methoxydibenzoylmethane, ethylhexyl salicylate, homosalate and combinations thereof.

15. The composition of claim 1, wherein (b) includes at least one water-soluble active ingredient selected from the group consisting of baicalin, ferulic acid, adenosine, resveratrol, ascorbic acid and derivatives, jasmonic acid and derivatives, hyaluronic acid and derivatives, hydroxypropyl tetrahydropyrantriol and combinations thereof, wherein the at least one water-soluble active ingredient is at a concentration by weight of from about 0.1% to about 10%, based upon weight of the sunscreen composition.

16. The composition of claim 1, wherein the sunscreen composition is stable, has a sun protection factor (SPF) of greater than or equal to 15.

17. The composition of claim 1, wherein the sunscreen composition exhibits a critical wavelength of greater than 370 nm.

18. A sunscreen composition comprising:
(a) an oil phase comprising:
(i) at least one silicone elastomer gel comprising an emulsifying crosslinked silicone elastomer having both silicone chain branches and alkyl chain branches swelled in a non-polar, volatile swelling agent having a Hansen solubility parameter, delta a, of less than or equal to about 5, the at least one silicone elastomer gel being present in an amount of from about 5% to about 10% by weight, based on the total weight of the sunscreen composition and the emulsifying crosslinked silicone elastomer being present in an amount of 20% to about 40% by weight, based on total weight of the silicone elastomer gel;
(ii) from about 14% to about 20% by weight of the sunscreen composition, of at least one organic sunscreen active; and,
(b) an aqueous phase comprising water; and
wherein the sunscreen composition is a homogenous, water-in-oil emulsion that is storage stable and includes one or both of a sun protection factor (SPF) of greater than or equal to 15 and exhibits a critical wavelength of greater than 370 nm.

19. A process for preparing the cosmetic composition according to claim 1, comprising:
mixing the oil phase;
mixing the aqueous phase; and
slowly adding the mixed aqueous phase to the mixed oil phase while mixing, forming a water-in-oil emulsion.

20. A method of imparting sunscreen benefits onto a keratinous substrate by applying the composition of claim 1 onto said keratinous substrate.
